(19) United States
(12) Patent Application Publication
(10) Pub. No.: US 2017/0340789 A1
(43) Pub. Date: Nov. 30, 2017

(54) CAVO-ARTERIAL PUMP

(71) Applicant: Yale University, New Haven, CT (US)

(72) Inventors: Pramod Bonde, Woodbridge, CT (US); John Valdovinos, Northridge, CA (US)

(21) Appl. No.: 15/606,217

(22) Filed: May 26, 2017

Related U.S. Application Data
(60) Provisional application No. 62/342,301, filed on May 27, 2016.

(51) Int. Cl. A61M 1/10; A61M 1/36; A61M 1/12; A61M 25/00

(52) U.S. Cl. CPC A61M 1/1049; A61M 25/0068; A61M 1/3653; A61M 1/1046; A61M 1/122; A61M 1/101; A61M 25/0043

(57) ABSTRACT

The present invention provides an intravascular right ventricular assist device, i.e., the cavo-arterial pump (CAP). Two prototypes of the CAP were developed, including a direct drive CAP and a magnetic drive CAP, demonstrating the feasibility of providing adequate pulmonary support and the feasibility of using axial magnetic couplings for contactless torque transmission from the motor shaft to the pump impeller. The magnetic drive CAP was able to operate up to 18.5 kRPM and produce a maximum flow rate of 1.35 L/min and a maximum pressure head of 40 mm Hg.

### CROSS-REFERENCE TO RELATED APPLICATIONS

This application claims priority to U.S. Provisional Patent Application No. 62/342,301, filed on May 27, 2016, the contents of which are incorporated by reference herein in its entirety.

### BACKGROUND OF THE INVENTION

Right ventricular (RV) dysfunction due to pulmonary hypertension, acute myocardial infarction, and left ventricular assist device-induced hemodynamic changes has limited the effectiveness of mechanical circulatory support therapy in heart failure patients. Right ventricular (RV) dysfunction can result as a sequelae of pulmonary hypertension, myocardial infarction, and acute/chronic volume or pressure overload conditions. Mechanical circulatory support (MCS) devices, specifically left ventricular assist devices (LVADs), have extended the lives of many adults suffering from end-stage congestive heart failure (HF). However, LVAD-induced right heart dysfunction is a problem that has limited the effectiveness of MCS therapy in the HF population (Dang et al., J Heart Lung Transplant. 2006, 25(1):1-6). Some researchers have reported up to 30-40% of heart failure patients with LVADs have developed some degree of right heart dysfunction regardless of the type of device used (pulsatile versus continuous flow; Patel et al., Ann Thorac Surg. 2008, 86(3):832-840). The majority of patients that develop right heart failure are relegated to drug therapy. Several groups have used currently available LVADs to support the RV with mixed results (Bernhardt et al., Eur J Cardiothorac Surg. 2015, 48(1):158-162; Potapov et al., ASAIO J. 2012, 58(1):15-18). Despite the potential of RVAD therapy, the development of right ventricular assist devices (RVADs) has lagged significantly compared to LVAD technology. An RVAD that can be deployed without a sternotomy and that can provide safe pulmonary circulatory support would be ideal for patients that develop right heart failure.

Percutaneous intravascular devices offer the potential to support the failing RV without the need for an extensive surgical procedure. Percutaneous blood pumps are devices that can be implanted via catheter-based procedures and are typically used clinically to provide partial support (2.5-5 L/min) for patients with acute HF. Recently, Stretch et al. showed that the use of percutaneous intravascular devices for short-term MCS in patients with acute HF has increased approximately 10 times between 2007 and 2011 (Stretch et al., Journal of the American College of Cardiology. 2014, 64(14):1407-1415). During this period, hospitals saw a decrease in mortality and morbidity and a decrease in hospital costs. Percutaneous pumps have already been successfully used as right ventricular assist devices in the setting of acute right ventricular failure (Kapur et al. The Journal of Heart and Lung Transplantation. 2011, 30(12):1360-1367; Cheung et al., J Heart Lung Transplant. 2014, 33(8):794-799). In these clinical studies, the percutaneous pump provided increased patient cardiac index, reduced patient central venous pressure, and mediated recovery of RV function. All of these effects facilitated RV recovery and eventual device explantation in some patients. Computer simulation studies also suggest that RVADs, in most circumstances, only need to provide a modest 1.5 to 2 L/min in additional flow to benefit patient hemodynamics (Punnoose et al., Progress in cardiovascular diseases. 2012, 55(2):234-243.e232). Despite this potential paradigm shift in RV dysfunction therapy, percutaneous pump technology is still limited to short-term use (a few hours) because of the need for a driveline to power the device and the need for purge sealing system that cools the pump motor and provides a seal between the motor-shaft and impeller interface (Butler et al., IEEE Trans Biomed Eng. 1990, 37(2):193-196; Rosarius et al., Artif Organs. 1994, 18(7):512-516; Siess et al., Artif Organs. 2001, 25(5):414-421). Together, the purging fluid line and driveline exit the patient's vasculature and limits patient mobility.

Thus, there is a need in the art for novel right ventricular assist devices (RVADs), in particular RVADs that can be deployed without a sternotomy and that can provide safe pulmonary circulatory support for patients that develop right heart failure. There is also a need in the art for novel RVADs featuring axial magnetic couplings which can help to eliminate the seal, and sealing system, typically needed to isolate the motor and bearings from blood contact. The present invention satisfies these unmet needs.

### SUMMARY OF THE INVENTION

In one aspect, the invention relates to an implantable device for transferring a bodily fluid between two anatomically distinct locations in a subject, comprising: a pump unit having an inflow port and an outflow port; at least one anchoring structure associated with the pump unit; and a conduit having first and second ends, the first end connected to the outflow port of the pump unit, and the second end having an outflow port. In one embodiment, the pump unit has a substantially cylindrical cross section, and a diameter between about 1 mm and about 20 mm. In another embodiment, the pump unit comprises a motor having a motor shaft, an impeller, a casing, and a diffuser. In one embodiment, the impeller is attached to the motor shaft. In another embodiment, the device further comprises a drive magnet and a following magnet, wherein the drive magnet is connected to the motor shaft, and the following magnet is connected to the impeller. In one embodiment, the device is a catheter-deliverable cavo-arterial pump (CAP). In another embodiment, the device is a catheter-deliverable right ventricular assist device (RVAD). In one embodiment, the anchoring structure comprises at least a strut comprising a nonferromagnetic flexible material. In another embodiment, the pump unit comprises a cable for transfer of power and data to and from the device. In another embodiment, the conduit comprises an optional cannula.

In another aspect, the invention relates to a method of assisting right ventricular circulation in a subject, comprising: placing the device of claim 1 in the vasculature of the subject, wherein the pump unit is anchored to the wall of the inferior vena cava (IVC) of the subject, and the outflow port of the conduit is placed in the main pulmonary artery of the subject; and directing blood flow through the device, from the IVC of the subject to the main pulmonary artery of the subject. In one embodiment, the conduit passes through the right atrium and the right ventricle of the subject. In one embodiment, the pump unit comprises a motor having a motor shaft, an impeller, a casing, and a diffuser, wherein the impeller is attached to the motor shaft. In another embodiment, the pump unit comprises a motor having a motor shaft, an impeller, a casing, a diffuser, a drive magnet, and a following magnet, wherein the drive magnet is connected to the motor shaft and the following magnet is connected to the impeller. In one embodiment, the pump unit comprises a cable for transfer of power and data to and from the device. In another embodiment, the conduit comprises an optional cannula. In another embodiment, the anchoring structure comprises at least a strut comprising a nonferromagnetic flexible material. In one embodiment, the blood flow is between about 0 and about 5 L/min. In another embodiment, the pressure head is between about 5 mmHg and about 100 mmHg. In another embodiment, the impeller speed is between about 5 kRPM and about 30 kRPM.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a schematic depicting the placement of an exemplary cavo-arterial pump (CAP).

FIG. 2 is a photograph of an exemplary device of the invention, the cavo-arterial pump.

FIG. 3 is a schematic depicting an exploded view of the direct drive CAP.

FIG. 4A is a schematic depicting an exploded view of the experimental prototype for testing magnetic couplings.

FIG. 4B is a finite element model for calculating the maximum torque transfer from the drive magnet to a following magnet separated by an air gap.

FIG. 4C is a graph of the torque magnitude at various air gap distances and magnetization offset angles.

FIG. 5 is a schematic depicting the experimental setup for the direct drive CAP.

FIG. 6A is a schematic depicting the experimental setup for the magnetically driven CAP.

FIG. 6B is a photograph of a prototype experimental setup for the magnetically driven CAP.

FIG. 7 is a chart depicting the results of a computational fluid dynamic model used to predict the pump performance curve.

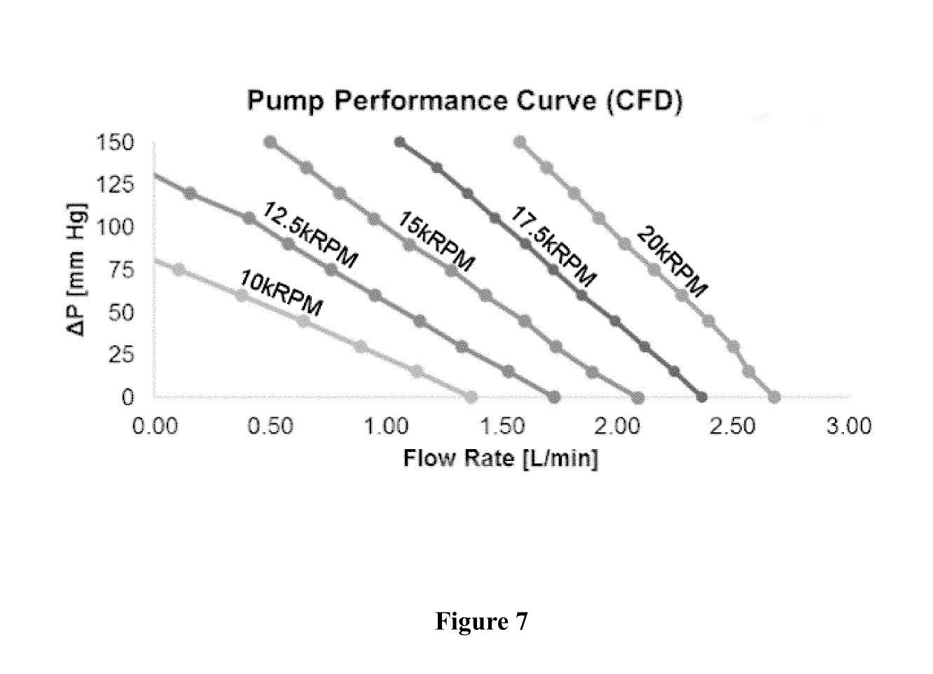

FIG. 8A and FIG. 8B, are a pair of charts depicting the experimental pressure-flow performance curves for the directly-driven CAP with water as the working fluid (FIG. 8A), and with blood analog (60% water, 40% glycerol) as the working fluid (FIG. 8B).

FIG. 9A and FIG. 9B, is a pair of charts depicting the experimental motor speed versus impeller speed results for CAP utilizing magnetic couplings with water as the working fluid (FIG. 9A), and with blood analog (60% water, 40% glycerol) as the working fluid (FIG. 9B).

FIG. 10A and FIG. 10B, is a pair of charts depicting the experimental flow rate as a function of motor shaft speed for CAP utilizing magnetic couplings with water as the working fluid (FIG. 10A), and with blood analog (60% water, 40% glycerol) as the working fluid (FIG. 10B).

FIG. 11A and FIG. 11B, is a pair of charts depicting the experimental pressure-flow performance curves for the magnetically-driven CAP with water as the working fluid (FIG. 11A), and with blood analog (60% water, 40% glycerol) as the working fluid (FIG. 11B).

### DETAILED DESCRIPTION

The invention relates in part to a cavo-arterial pump (CAP), functioning as a right ventricular assist device (RVAD), which is an intravascular blood pump designed to provide pulmonary circulatory support for patients that develop RV dysfunction, in particular LVAD-induced RV dysfunction. The pump features either a direct drive pump mechanism, or a magnetic drive pump mechanism. The magnetic drive mechanism eliminates the need for an external purge seal line by utilizing permanent magnet magnetic bearings or magnetic couplings, enabling the development of fully implantable intravascular pumps.

An intravascular pump of the invention can provide sufficient pulmonary support, i.e., up to about 2.25 L/min. In a magnetic drive pump of the invention, including magnets with an about 90 degree offset separated by an about 2.5 mm gap, the coupling can provide up to about 6 mNm of torque. The couplings can be spaced up to 4 mm apart before torque transmission falls below the motor output. The magnetic drive CAP was able to operate at up to about 18.5 kRPM, and produce a maximum flow rate of about 1.35 L/min and a maximum pressure head of about 40 mm Hg. In addition, computational fluid dynamic (CFD) simulations show that the pump can provide flow between 1.4-3 L/min of flow at venous pressures (0-30 mmHg) when the motor is run between 10 kRPM and 22 kRPM.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

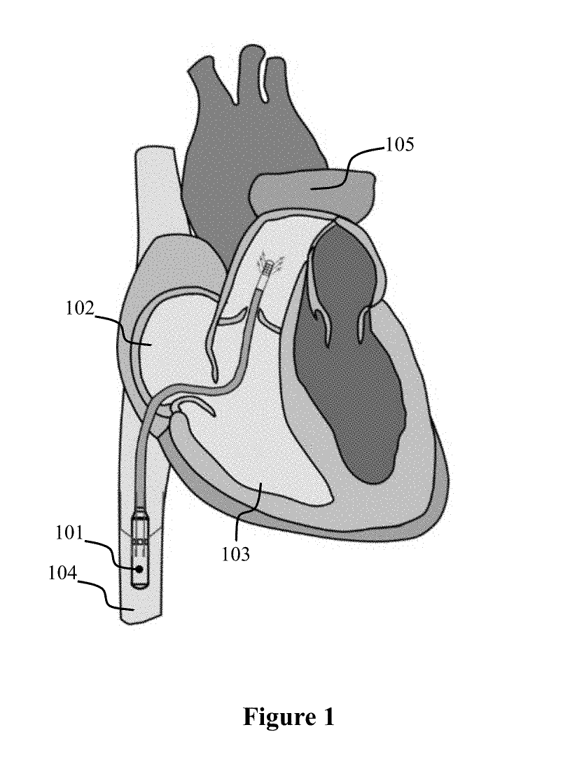

In one aspect, the invention relates to a minimally-invasive cavo-arterial pump device that can be positioned within the body of a subject to aid in the movement or pumping of a bodily fluid. For example, in certain instances, the device provides for movement of blood, urine, sweat, air, and the like. In a particular embodiment, the device aids or replaces ventricle function of the heart by moving blood past the right or left ventricle into the pulmonary or systemic circulation, respectively. In one embodiment, the placement of device 101 is as depicted in FIG. 1, moving blood from inferior vena cava (IVC) 104, bypassing right atrium 102 and right ventricle 103, and directly into main pulmonary artery 105.

In one embodiment, the invention provides right ventricular assist devices (RVADs) configured for minimally-invasive percutaneous delivery to the implantation site. The devices are capable of providing long-term support with overall hemodynamic performance and durability superior and comparable to current conventional therapeutic approaches to right ventricular assistance. The devices are constructed of durable materials allowing for long-term use. A device of the invention generally has dimensions that allow for its insertion and guidance through a blood vessel.

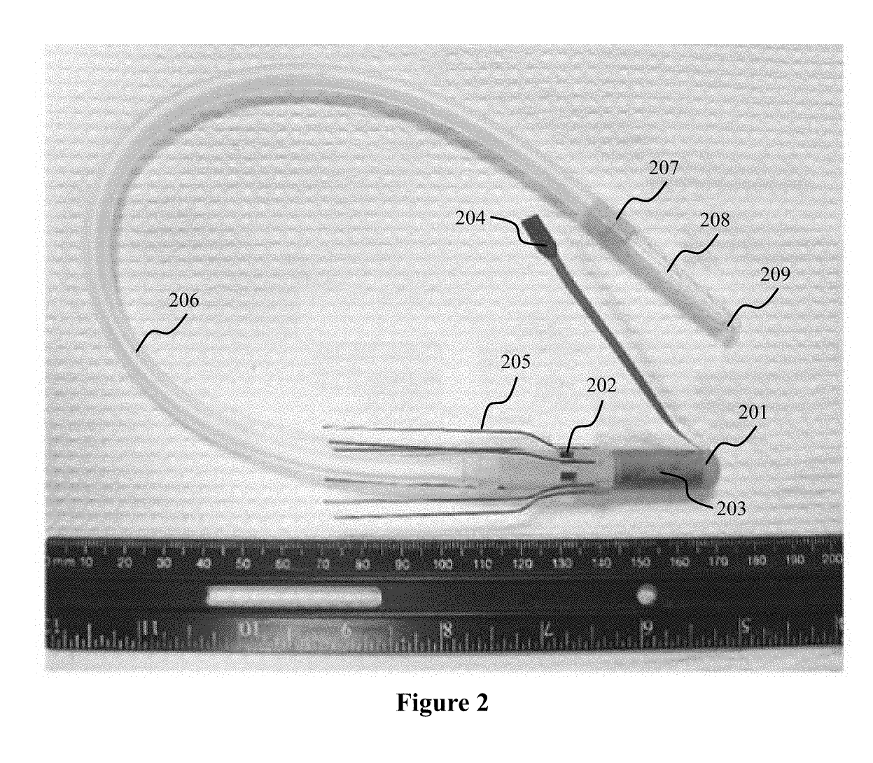

As shown in FIG. 2, in one embodiment, the device of the invention comprises pump unit 201. The pump unit includes a casing having inlets 202 for the inflow of blood from IVC 104. Pump unit 201 further includes motor 203, having attached an optional power strip 204. Attached to the periphery of the pump casing or the motor is one or more anchoring structures 205 for positioning the pump in the IVC. Anchoring structure 205 may comprise one or more struts, legs, hooks, loops, barbs, or any other protrusion capable of attaching to the vessel wall and anchoring pump unit 201 in IVC 104. In some embodiments, the one or more anchoring structures 205 are of the same size that are evenly spaced around pump unit 201, or may be spaced irregularly as needed to conform to the shape of IVC 104. While the embodiment of FIG. 2 shows six anchoring structures 205, it should be appreciated that the number of anchoring structures 205 can include 1, 2, 3, 4, 5, 6, 7, 8, 9, 10 or more than 10 anchoring structures 205. Anchoring structure 205 may interface with pump unit 201 in any suitable way, including via adhesion, electrical energy, or over-molding. Alternatively, anchoring structure 205 may be formed integral to the pump unit 201 through techniques including but not limited to, molding, laser fabrication, or formation from other known manufacturing techniques. In some embodiments, anchoring structures 205 are deployable, where they can be triggered to transition from a first state to a second state. For example, in some embodiments, anchoring structures 205 are responsive to a mechanical, electrical, or biological stimuli to move from a retracted state to an extended state and vice-versa, in order to facilitate ease of delivery. In another embodiment, pump unit 201 and anchoring structures 205 are compressible into a substantially cylindrical configuration, such that the device fits within a catheter having a lumen, for delivery to or retrieval from IVC 104.

As depicted in FIG. 2, in some embodiments, anchoring structure 205 comprises an anchoring strut or leg. The one or more anchoring struts or legs can be either, or both, distally or proximally leaning. As readily apparent from FIG. 2, in one embodiment anchoring struts are leaning proximally. In some embodiments, one or more anchoring struts or legs comprise one or more hooks, barbs, or hoops that engage with the vessel wall. In some embodiments, the struts are flexible and can collapse inward toward pump unit 201 when in a compressed state. In a relaxed state (as shown in FIG. 2), the struts may expand away from pump unit 201.

Further attached to pump 201 is fluid conduit 206, for example a flexible tube, for blood transport, conduit 206 ending with outflow port 207 for blood delivery into main pulmonary artery 105. The flexible tube can have optional terminal cannula 208 also having outflow port 209. In one embodiment, pump 201 is about 10 mm in diameter and about 65 mm in length.

It should be appreciated that the casing, tubing 206, outflow port 207, and optional cannula 208 can be composed of any material, such as medical grade alloys or polymers. In some embodiments anchoring structures 205 can be composed of a nonferromagnetic, flexible, shape memory material, such as nitinol, a composite of nickel and titanium known for its superelasticity and ability to expand to a different shape. For example, in one embodiment, the struts are configured to expand at a temperature threshold at or near body temperature. It should be appreciated that any rigid, yet flexible material may be used, such as a medical grade alloy or polymer, so that struts can be compressed inwardly toward the body of pump 201 in a compressed state, creating an expanding bias which can be restrained for example by a delivery catheter. Once the restraint is removed, for example by removing the body of pump 201 from the delivery catheter, the expanding bias forces struts to return to their relaxed, expanded state. The medical grade materials described herein may also include an anti-thrombogenic coating or admixture to reduce the incidence of thrombus buildup, promoting hemocompatibility and the maintenance of high blood flow rates pass the pump. The material may also include a coating comprising an immunosuppressant, e.g., rapamycin (sirolimus).

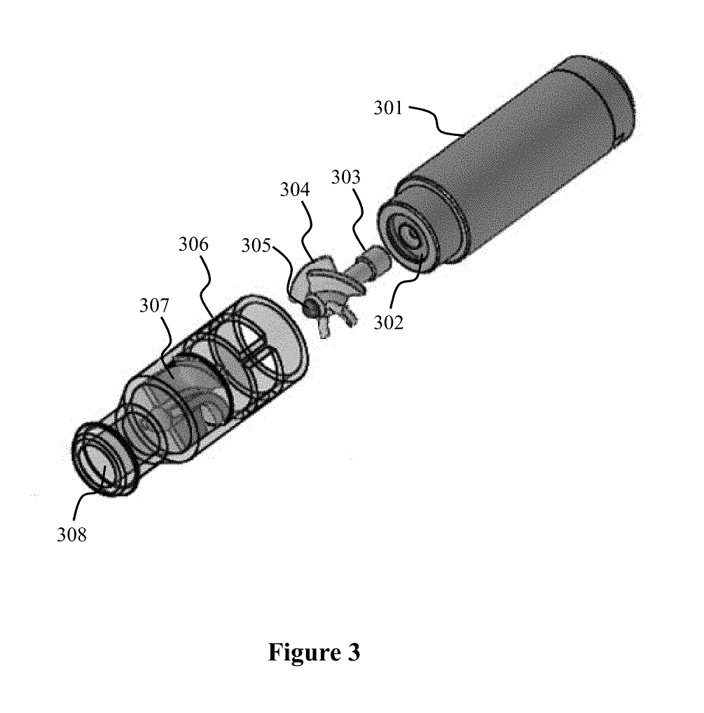

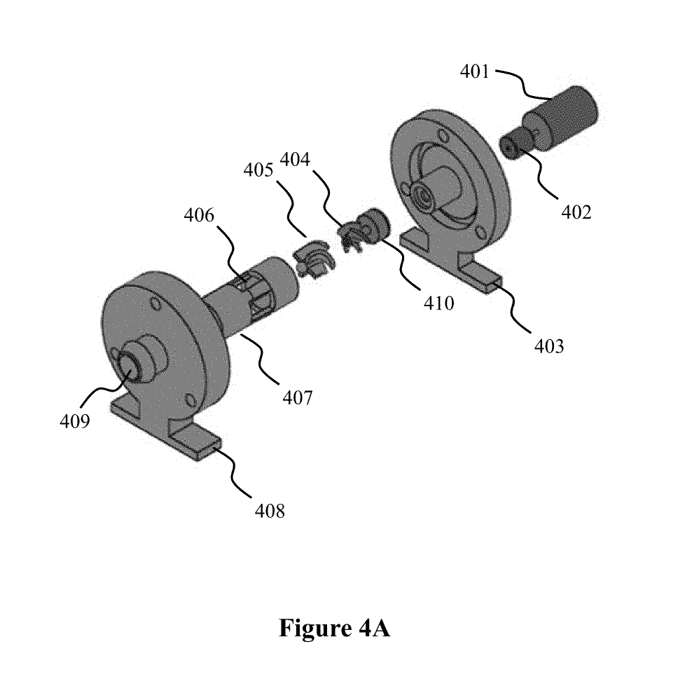

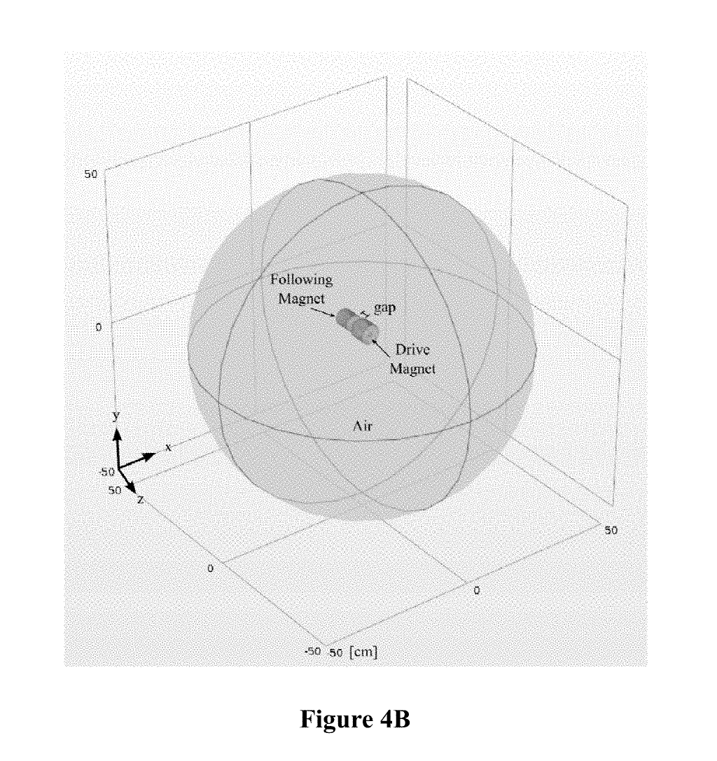

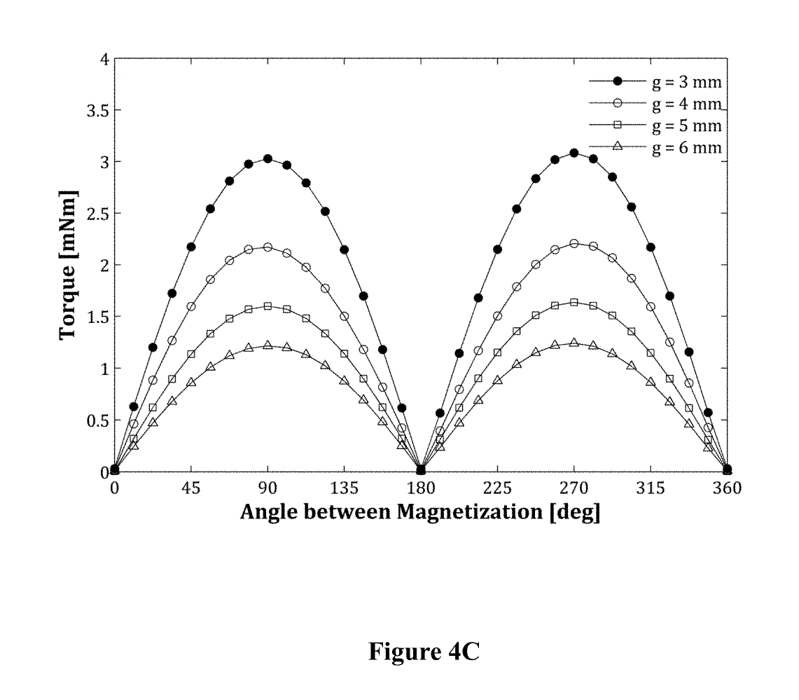

The cavo-arterial pump of the invention can be designed to work with either a direct drive mechanism pump (FIG. 3), or with a magnetic drive mechanism pump (FIG. 4A). Any suitable motor can be used in either the direct drive mechanism pump, or the magnetic drive mechanism pump. In one embodiment, the motor is a brushless in-runner motor. The motor can be substantially cylindrical and have a diameter between about 1 mm and about 20 mm. In one embodiment, the diameter of the motor is about 10 mm. The motor is connected, either directly or magnetically, to an impeller (304 or 404) having four blades. As readily apparent, the impeller can have any suitable number of blades. For example, in other embodiments, the impeller has two blades, three blades, five blades, six blades, or the like. The impeller can have a diameter between 1 mm and 20 mm, and a length between about 1 mm and about 20 mm. In one embodiment, the impeller is a 4-blade axial impeller which is 7.5 mm in diameter and 4 mm in length. The blades may be located at the proximal end of the impeller, the distal end of the impeller, or along the entire length of the impeller.

In either the direct drive mechanism pump (FIG. 3), or the magnetic drive mechanism pump (FIG. 4A), the impeller (304 or 404) is housed inside a pump casing. The pump casing surrounds the sealed motor (301 or 401), drive shaft (303) and impeller (304 or 404). The casing can have four side inlets 406 for the inflow of the blood from the IVC, and one outlet (308 or 409) for the outflow of the blood into transport tube 206 and toward the pulmonary artery 105. As readily apparent, any number of inlets or outlets can be used. Next to the impeller, in the direction of the blood flow, is a diffuser (307 or 405) attached to the pump casing. The diffuser enables pressure recovery from rotating impeller (304 or 404) through outlet (308 or 409). Upon rotation of the impeller, as driven by either the direct drive shaft 303, or the magnetic drive 402, the blood is pumped out of the pump unit via the fluid outlets of the housing.

As shown in the exploded view in FIG. 3, a direct drive mechanism pump includes motor 301 having a radial bearing 302, and a motor shaft 303 with an impeller 304 attached to it, the impeller further having an axial bearing 305. Impeller 304 can be attached to the motor shaft 303 by any suitable method known in the art. In one embodiment, the impeller is glued to the motor shaft using an epoxy adhesive. In one embodiment, the motor shaft is hermetically sealed from the working fluid, while in another embodiment, the motor shaft is not hermetically sealed from the working fluid. In one embodiment, the motor and drive shaft are completely sealed from fluid, which eliminates the need for a purge fluid line present in current temporary percutaneous devices to keep blood from entering the motor. The impeller is outside of the sealed region of the pump, thus allowing the impeller to come into contact with the fluid, i.e., venous blood. In one embodiment, a sapphire ring bearing is used for radial stabilization of the impeller, and a sapphire hemisphere and cup are used as axial bearings. As readily apparent, any suitable type of ring, hemisphere, and cup bearings can be used.

In a preferred embodiment, a pump of the invention includes an axial magnetic coupling utilizing permanent magnets, for example neodymium permanent magnets. An axial magnetic coupling offers the potential to eliminate the purge seal needed in intravascular pumps previously known in the art. As shown in the exploded view in FIG. 4A, a magnetic drive mechanism pump includes a motor 401, for example an in-runner motor, having a diametrically magnetized drive magnet 402 attached to the shaft of the motor. Compared to the direct drive mechanism pump, impeller 404 of the magnetic drive mechanism pump is modified to encase a 2-pole diametrically magnetized magnet 410, known as the following magnet. As readily apparent, both drive magnet 402 and following magnet 410 can have any suitable shape, including, but not limited to, spherical, cylindrical, rectangular, polygonal, arc-shaped, ring-shaped, and the like. As readily apparent, any number of magnetic poles can be used. For example, 4 poles, 6 poles, 8 poles, 10 poles, or the like, can be used in either, or both, drive magnet 402 and following magnet 410. In another embodiment, a radial magnetic coupling can be used in either, or both, drive magnet 402 and following magnet 410.

Similarly to direct drive pump, impeller 404 and diffuser 405 in the magnetic drive pump have a sapphire hemisphere and cup as axial bearings, respectively, but as readily apparent, any suitable type of hemisphere and cup bearings can be used. As readily apparent, the magnetic drive pump operates by the magnetic field coupling of magnets 402 and 410. When motor 401 rotates, drive magnet 402 will engage following magnet 410 through a magnetic field, and as a result following magnet 410 will rotate attached impeller 404. As readily apparent, the gap distance between drive magnet 402 and impeller following magnet 410 can vary, and is generally between about 0.05 mm to about 20 mm. In one embodiment, the gap distance between drive magnet 402 and impeller following magnet 410 is about 1 mm. In another embodiment, the gap distance between drive magnet 402 and impeller following magnet 410 is about 2.5 mm. In another embodiment, the gap between impeller magnet 410 and drive magnet 402 is reduced to the minimum limit allowed by fabrication tolerances.

In one embodiment, the pump of the invention has a motor capable of achieving various rotation speeds between 5 and 30 kRPM (thousands of rotations per minute). In various embodiments, the motor can rotate at 10.7 kRPM, 11 kRPM, 14.5 kRPM, 14.7 kRPM, 16 kRPM, 16.7 kRPM, 17.5 kRPM, 20 kRPM, and 24 kRPM, or any other suitable speed. As readily apparent, in a direct drive mechanism pump, the rotational speed of the impeller is identical to the rotational speed of the motor shaft.

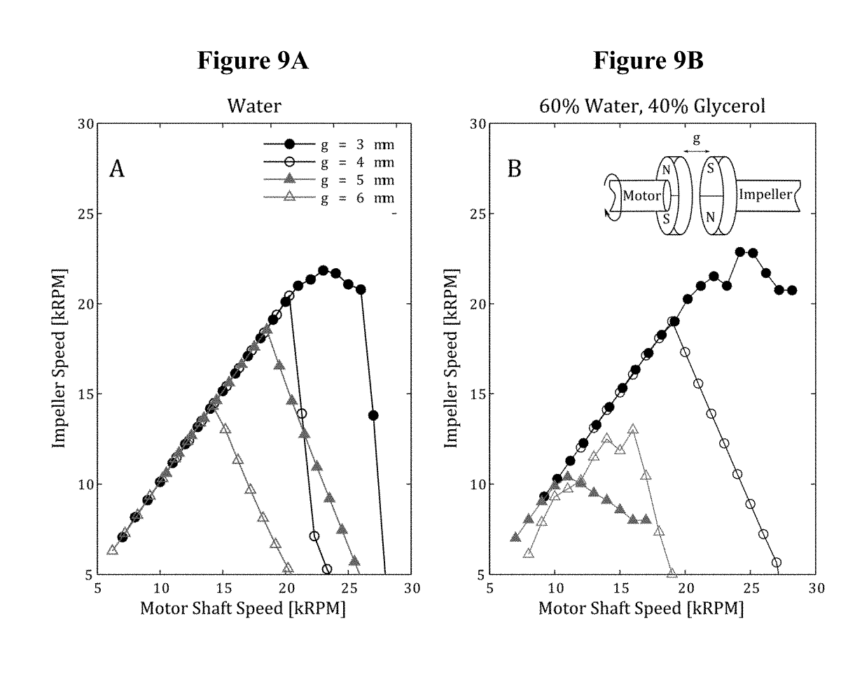

For the magnetic drive mechanism pump, the rotational speed of the impeller is equal or less than the rotational speed of the motor shaft, and the relationship between the rotational speed of the impeller and the rotational speed of the motor is influenced by the gap distance between the drive magnet and the following magnet, and the physical properties of the liquid being pumped. For example, for a 3 mm separation, while pumping water, the impeller speed matches the motor rotational speed up to 21 kRPM, and above 21 kRPM the impeller rotational speed decreases with increasing motor shaft speed. Similarly, while pumping water, the maximum rotational speed in which the impeller matches the motor shaft speed in a magnetic drive pump is 20.3, 18.5, and 14.3 kRPM for 4 mm, 5 mm, and 6 mm gap distance magnet separation, respectively (FIG. 9A). FIG. 9B shows the impeller rotational speed as a function of motor shaft speed when the pump is submerged in water-glycerol solution. The maximum rotational speed in which the impeller matches the motor shaft speed is 21 kRPM, 19 kRPM, and 11 kRPM for a 3 mm, 4 mm, and 5 mm magnet separation, respectively. When the impeller and drive magnets are separated by 6 mm, the impeller rotational speed is consistently slower than the motor shaft speed (horizontal shift in line). In one embodiment, a one to one matching between the motor shaft speed and the impeller rotational speed is provided at least up to 18 kRPM.

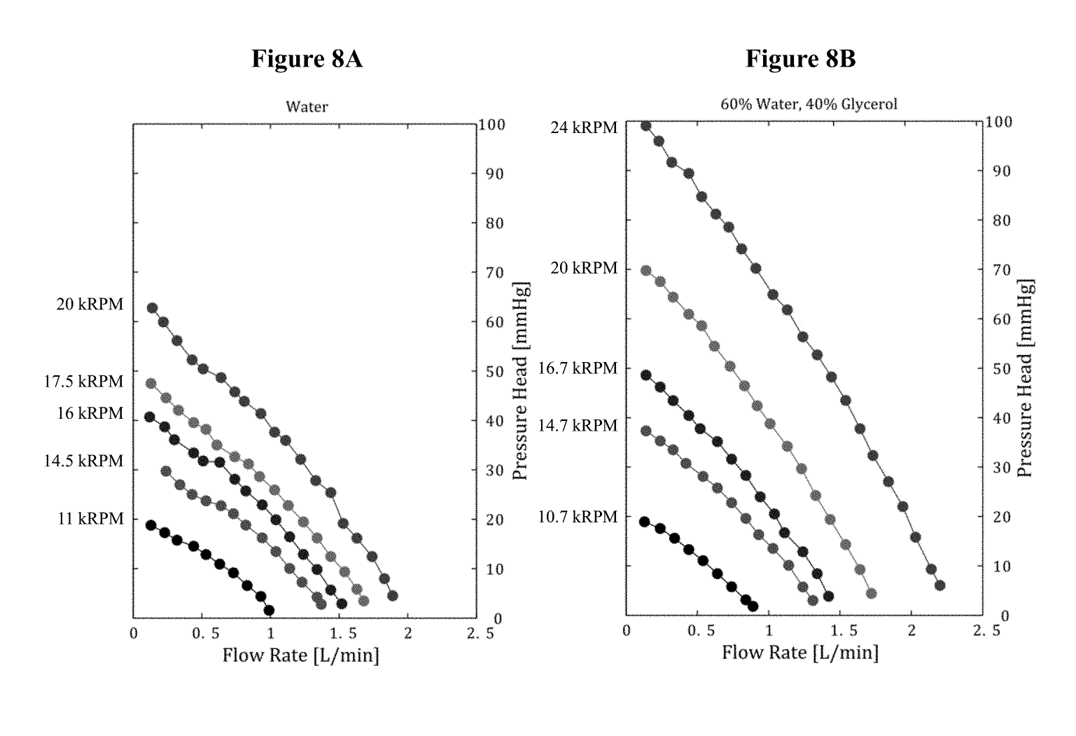

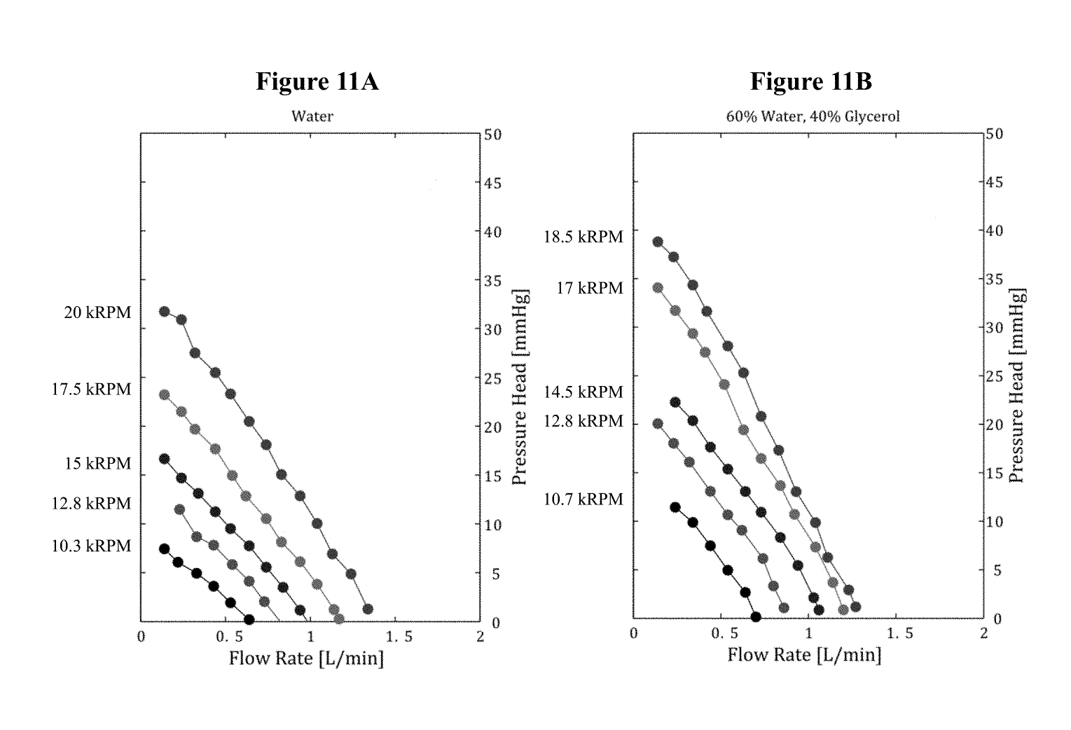

In either the direct drive mechanism pump (FIG. 8), or the magnetic drive mechanism pump (FIG. 11), the pressure head produced by a pump of the invention as a function of pump flow rate varies at various motor shaft speeds. In one embodiment, a pump of the invention can generate any flow rate between about 0 and about 5 L/min (liters per minute). In another embodiment, a pump of the invention can generate a pressure head between about 5 and about 100 mmHg.

In one embodiment, the device of the invention includes a power cable operably connected to the motor. In certain embodiments, the power cable, lead, or line, can be externalized from the device to outside of the body using known techniques. For example, in one embodiment, the power cable can be guided from the device through the superior vena cava and into the subclavian vein to an area over the right or left side of the chest, where a small incision can be made to retrieve the cable. In one embodiment, the pump can be powered via a transfemoral lead that exits the patient's femoral artery. In another embodiment, the power line exits the brachiocephalic vein, while the controller, backup battery, and a wireless powering coil resides in the infra-clavicular pocket.

The device of the invention can be operated with both wired and/or transcutaneous energy transfer (TET) power delivery systems. For the implementation of TET power delivery, a small superficial pocket is created just underneath the skin where a TET coil can be placed and connected to the power cable of the device. Exemplary TET power delivery systems, including systems that wirelessly deliver power to implantable devices, are described in U.S. patent application Ser. Nos. 13/843,884 and 14/213,256, each of which are incorporated by reference in their entirety.

In certain embodiments, the device of the invention is operably connected to a pump controller. The pump controller may be located exterior to a patient, or implanted within the patient. In certain embodiments, the pump controller delivers and receives signals from the device relating to function of the pump unit of the device. For example, the controller may provide signals relating to the control of pump speed, desired flow rate, type of flow produced (pulsatile vs. continuous), and the like. The controller may be directly wired to the device of the invention or may communicate wirelessly to the device.

In some embodiments, the device of the invention is controlled by an implantable controller that is sized and shaped to be implanted within the body of the user. The controller may comprise a power supply, or alternatively may be powered externally by a separate wired or wireless power source positioned outside the body of the user. In one embodiment, the invention may be powered by a wireless power system, such as a system as described in U.S. Pat. No. 8,299,652; U.S. Patent Application Publication No. 2013/0310630; Sample et al., 2011, IEEE Transactions, 58(2): 544-554; and Waters et al., 2012, Proceedings of the IEEE, 100(1): 138-149, the entire disclosures of which is incorporated by reference herein in their entireties.

In some embodiments, the controller is communicatively connected to an external control unit, which may comprise a smartphone, a desktop, a tablet, a wristwatch, or any suitable computing device known in the art. In addition to exercising control over the various functions of the CAP, the controller may receive data from one or more sensors. Examples of such data include an EKG signal, the current pump speed of the CAP, the current flow rate within the CAP, the power consumption of the CAP, pulse oximetry, or any other information relevant to the function of the CAP. In some embodiments, some or all of the collected data is presented as part of a user interface (UI) of the external control unit. In some embodiments, the UI may provide the user with the ability to modify the function of the controller, display information related to historical or real-time functionality of the CAP, and/or display historical or real-time information related to the user's cardiac function.

As would be understood by those skilled in the art, the external control unit may be directly connected via wires or wirelessly connected via any suitable radio-frequency, optical, or other wireless communication standard. In some embodiments, the external control unit may be physically far removed from the CAP and only in indirect communication with the CAP and/or the implantable controller, connected via one or more wireless networks, Ethernet switches, or the Internet. In some embodiments, control signals transmitted from the external control unit to the implantable controller are encrypted.

The present invention comprises a method of promoting the movement or flow of a body fluid. The method may be used to aid in the movement or pumping of any body fluid in any location within the body. For example, in certain embodiments, the method comprises delivery and implantation of the device described herein into the IVC to promote pumping of blood to the pulmonary artery. The device thereby provides long term RVAD function. In one embodiment, the method comprises inserting the device into the vasculature, and guiding the device through the vasculature to the implantation site. In some embodiments, the method comprises inserting a delivery catheter, loaded with the device of the invention, into the vasculature, and guiding the catheter and device to the implantation site. In one embodiment, the method comprises releasing the device from the delivery catheter at the implantation sit. In one embodiment, releasing the device from the catheter allows for one or more anchoring structures to expand into its relaxed state to allow for engagement of the vessel wall. In one embodiment, the method comprises anchoring the pump unit in the vessel wall in the terminal portion of the IVC. In one embodiment, the method comprises guiding the fluid conduit to the right atrium via the cavo-atrial opening, to the right ventricle via the tricuspid valve, and to the pulmonary artery via the pulmonary valve, such that the outflow port resides in the lower portion of the pulmonary artery. However, the device may be inserted at any suitable access site.

In one embodiment, the method comprises sending a signal to the pump unit of the device to start the motor and set the rotation speed. In another embodiment, the method comprises setting the rotation speed based on a set of sensor inputs measured by the device or other implanted or external devices. In one embodiment, the method comprises sending a signal to the pump unit of the device to stop pumping based on one or more sensor inputs. In one embodiment, the method comprises intermittently starting or stopping the rotation of the pump. In some embodiments, the method comprises running the pump continuously, but varying the speed of the pump over time according to a pre-determined pattern. The method of the present invention may further comprise adjusting the speed of the pump motor based on sensor data related to the performance of the pump. For example, in response to a measured impeller rotation rate provided by a hall effect sensor or other rotation speed sensor, a controller might adjust the driven speed of the motor in order to optimize efficiency.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: CAP Design and Fabrication

The intravascular pump designed is intended to provide partial circulatory support (2.5-3 L/min) to patients with LVAD-induced right ventricular dysfunction. The intravascular pump 101, which is called the cavo-arterial pump (CAP), would sit in the inferior vena cava 104 and propel venous blood to the main pulmonary artery 105 (FIG. 1). Preliminary sizing of the pump impeller and speed of operation were determined by a combination of fabrication tolerances and the general design criteria for turbomachinery (Stepanoff, Centrifugal and Axial Flow Pumps: Theory, Design, and Application. Krieger Publishing Company; 1957). In this design iteration, the CAP was designed to produce 2.5 L/min against at 30 mm Hg pressure head for right ventricular support. The impeller diameter was set to 7.5 mm. The specific work of this pump was calculated using:

$$y = \frac{Δ P}{ρ} \qquad ( Equation 1 )$$

where y is the specific work, ΔP is the pressure head across the pump, and p is the density of blood. The specific work in this design was calculated to be 3.9 m²/s².

The specific diameter was also calculated using:

$${D}_{s} = 1.054 d \frac{{y}^{1 / 4}}{\sqrt{Q}} \qquad ( Equation 2 )$$

where Dₛ is the impeller specific diameter, d is the impeller diameter, and Q is the desired flow rate. For a diameter of 7.5 mm and a flow rate of 2.5 L/min, the specific diameter calculated is 1.72. Using a Cordier diagram, the specific speed, Nₛ, was found to be 2 for this design.

Lastly, the rotational speed required to produce 2.5 L/min against a 30 mm Hg pressure head using a 7.5 mm impeller was calculated using:

$$n = \frac{{N}_{s} {y}^{3 / 4}}{2.108 \sqrt{Q}} \qquad ( Equation 3 )$$

Thus, the impeller speed required to produce 2.5 L/min against a 30 mm Hg pressure head is 24 kRPM. An AC motor capable of achieving rotational speeds above 24 kRPM was chosen for device fabrication.

Two pump prototypes were designed and fabricated. A direct drive pump, in which the impeller was attached directly to the motor shaft was fabricated. In addition, the same design was adapted to use a magnetic drive mechanism. Both designs consist of a brushless 10 mm diameter in-runner motor (Turnigy 1015, Hobby King USA LLC, Lakewood, Wash., USA), 4-blade impeller and diffuser, and 10 mm outer diameter pump housing with 4 side inlets and one outlet. The impeller and diffuser were designed on ANSYS® BladeModeler and converted to three-dimensional models in ANSYS® DesignModeler.

The computer aided design (CAD) model of the direct drive pump is shown in FIG. 3. All parts were fabricated using the Objet30 Pro 3D printer (Stratasys Ltd., Eden Paraire, Minn., USA). This pump prototype consists of a 4-blade axial impeller 304 which is 7.5 mm in diameter and 4 mm in length. The impeller was epoxied to the motor shaft 303, which was not hermetically sealed from the working fluid. Even though the motor was submerged in fluid, it still functioned properly during experimental testing. A sapphire ring bearing 302 is used for radial stabilization of the impeller. A sapphire hemisphere and cup were used as axial bearings 305. The diffuser 307 was attached to the pump housing to enable pressure recovery from the rotating impeller 304 through the outlet 308. The prototype, shown in FIG. 2, is 10 mm in diameter and 46 mm in length.

A CAD model of the magnetic drive pump is shown in FIG. 4A. The parts, like the direct drive CAP, are 3D printed using the Objet30 Pro. The impeller 404 and diffuser 405 blade and hub geometries are the same as direct drive pump. The impeller 404 was modified to encase a 5 mm diameter and 5 mm long 2-pole diametrically magnetized neodymium iron boron (NdFeB) magnet, known as the following magnet. A drive magnet 402, 6 mm in diameter and 5 mm long, was attached to the shaft of the in-runner motor 401. A first stand 403 was fabricated to isolate the motor 401 and drive magnet 402 from the working fluid and to hold the sapphire radial bearing. A second stand 408, with an integrated pump housing 407, enclosed the diffuser 405. The entire setup was attached to an acrylic tank and sealed with epoxy.

A finite element model of two permanent magnet couplings was created on COMSOL Multiphysics® software (Burlington, Mass., USA) to estimate the range of torque values needed to rotate the impeller across an air gap. The model, shown in FIG. 4B, consists of two coaxial (along the z-direction) NdFeB magnets separated by an air gap. Dimensions of the magnets match the dimensions of those used in the CAP design. The magnetic polarities of the NdFeB magnets were assigned using a magnetic polarization vector with magnitude equivalent to the remnant polarization, Mᵣ, which was set to 1.45 Tesla. The magnetization direction for the following magnet was fixed along the x-direction to mimic a diametrically magnetized polarity. The magnetization direction of the drive magnet, relative to the stationary magnetization, was rotated at various angles, θ, within the x-y plane. The x and y-magnetization components were defined using:

Mₓ=Mᵣ cos(θ)

M_{y}=Mᵣ sin(θ)

A parametric sweep was carried out in which the gap between the magnets were changed from 3 mm to 6 mm (in 1 mm steps) and the angle, θ, was varied from 0° to 360° in 22.5° steps. The torque from the following magnet to the drive magnet was calculated for all these parameters. The torque calculations represent the maximum torque that can be transmitted by the magnetic couplings across the gap. Air was used as the surrounding medium. The wall separating the magnets and the working fluid were not taken into consideration in this model. The model consisted of 344,000 mesh elements.

The torque magnitude calculated from the finite element model at various angles and gap distances is shown in FIG. 4C. When the magnetization directions of the drive and following magnets are parallel (0° and 360° angle) or antiparallel (180° angle), the drive magnet exerts no torque on the following magnet regardless of the gap distance. When the magnetizations are perpendicular (90° or 270° angle), the drive magnet exerts maximum torque on the following magnet. The maximum torque magnitude is a function of the gap distance between the coupling magnets. For example, at 3 mm separation, the maximum amount of torque that can be transferred is 3 mNm. At 6 mm separation, at most, 1.2 mNm of torque can be transferred. Thus, the range for power transmission, in terms of torque, is limited by the gap distance between the coupling magnets and the orientation of the magnetic polarizations.

### Example 2: Direct Drive CAP

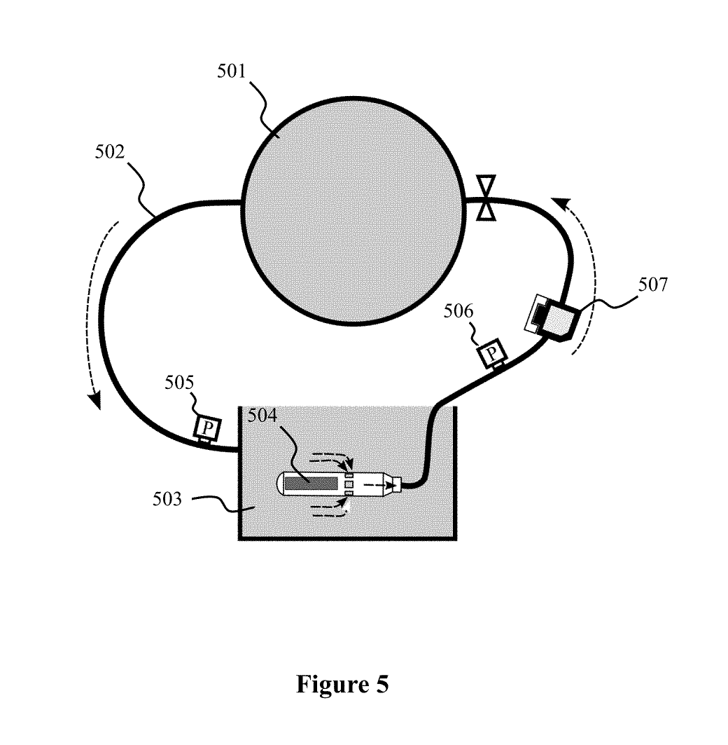

The direct drive CAP was tested on a bench-top flow loop to test the performance. The flow loop, shown in FIG. 5, consists of a reservoir 501, flexible tubing 502, and a submersion tank 503 for pump 504. Reusable blood pressure transducers 505 and 506, MLT0380, (AD Instruments, Dunedin, New Zealand) were used to measure the tank pressure and pump outlet pressure. An ultrasonic flow sensor, ME8PXL, and flow meter 507, TS410 (Transonic Systems Inc., Ithaca, N.Y., USA) were used to measure the pump flow rate. A gate valve located at the pump outlet was used to modulate the outlet resistance and increase afterload. Motor shaft speed was set with a sensorless motor drive (S48V5A, Koford Engineering LLC., Winchester, Ohio, USA) and external potentiometer. Two different working fluids were used. The first was water and the second was a 40% by volume glycerol and 60% by volume water solution to mimic the viscosity of blood. Viscosity was not specifically measured in these experiments. However, the same blood analog was used across all tests. CAP 504 was dunked into submersion tank 503 and run at various speeds under various outlet resistances. The pressure differential generated by the pump was calculated as the outlet pressure minus the tank pressure.

The performance of the direct drive CAP at different speeds in water is shown in FIG. 8A. The pressure head produced by the pump as a function of pump flow rate is displayed at various motor shaft speeds. For increasing pressure head, the flow rate produced by the pump decreases almost linearly. At 20 kRPM, the direct drive CAP was able to produce a maximum flow rate of 1.9 L/min and a maximum pressure head of 65 mm Hg. FIG. 8B shows the pump performance in the glycerol-water solution. At 20 kRPM the direct drive CAP was able to produce a larger stall pressure of 70 mm Hg but a lower no-afterload flow rate of 1.7 L/min. At 24 kRPM, the maximum speed the pump could reliably operate, the CAP produced a stall pressure of 100 mm Hg and a maximum flow rate of 2.2 L/min.

The direct drive CAP is capable of producing sufficient partial circulatory support in the pulmonary circulation of a right heart failure patient. As seen in FIG. 8B, the pump is able to produce 1.8-2.25 L/min flow rate for pressure heads varying from 0 mm Hg (during right ventricular systole) to 30 mm Hg (right ventricular diastole) when operated at 24 kRPM. For patients with pulmonary hypertension, where the systolic pressure can reach up to 60 mm Hg, the pump can provide between 1.5-2.25 L/min when operated at 24 kRPM. Thus, for the first design iteration, the CAP is suited to work as an effective partial support right ventricular assist device. It is important to note that the highest rotational speed achievable with this motor and pump design was 24 kRPM. Other designs will aim to rotate the impeller up to 30 kRPM for more flow output and reduce the overall diameter of the pump from 10 mm (30 Fr) to 7 mm (21 Fr).

### Example 3: Magnetic Drive CAP

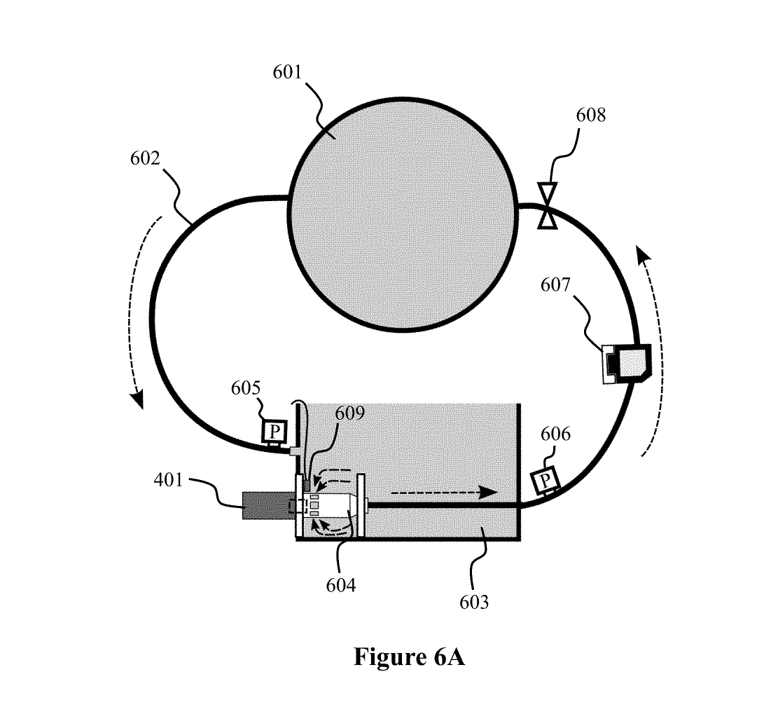

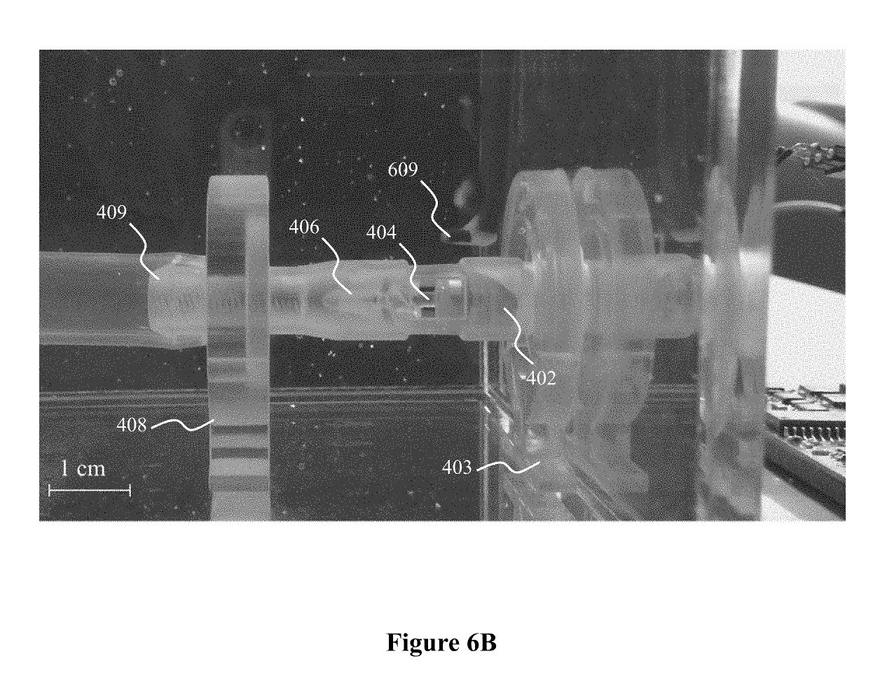

A second setup was fabricated to test the CAP driven with axial magnetic couplings. The magnetic drive CAP was tested on a bench-top flow loop similar to the direct drive CAP. The flow loop, shown in FIG. 6A, consists of a reservoir 601, flexible tubing 602, and an acrylic submersion tank 603 for pump 604. Reusable blood pressure transducers 605 and 606 were used to measure the generated pump pressure differential. An ultrasonic flow sensor 607 was used to measure the pump flow rate. A gate valve 608 was used to modulate pump afterload. Motor shaft speed was set with a sensorless motor drive and potentiometer. Shaft speed was measured from the motor drive via an encoder output. Impeller speed was measured with a bipolar hall-effect sensor 609 (SS40A, Honeywell International Inc., Morristown, N.J., USA) located above the pump inlet window and the impeller follower magnet. The gap distance between the drive magnet and impeller following magnet was modified. Water and water/glycerol solution were used as the two working fluids. A photograph of the experimental setup is shown in FIG. 6B.

The effectiveness of the magnetic coupling in the magnetically-driven CAP in water is shown in FIG. 9A. The measured impeller rotational speed is compared to the motor shaft speed for various impeller magnet to drive magnet gaps. For a 3 mm separation, the impeller speed matches the motor rotational speed up to 21 kRPM. Above 21 kRPM, the impeller rotational speed decreases with increasing motor shaft speed. Similarly, the maximum rotational speed in which the impeller matches the motor shaft speed is 20.3, 18.5, and 14.3 kRPM for 4, 5, and 6 mm magnet separation respectively. FIG. 9B shows the impeller rotational speed as a function of motor shaft speed when the pump is submerged in a blood analog (water-glycerol) solution. The maximum rotational speed in which the impeller matches the motor shaft speed is 21, 19, and 11 kRPM for a 3, 4, and 5 mm magnet separation. When the impeller and drive magnets are separated by 6 mm, the impeller rotational speed is consistently slower than the motor shaft speed (horizontal shift in line).

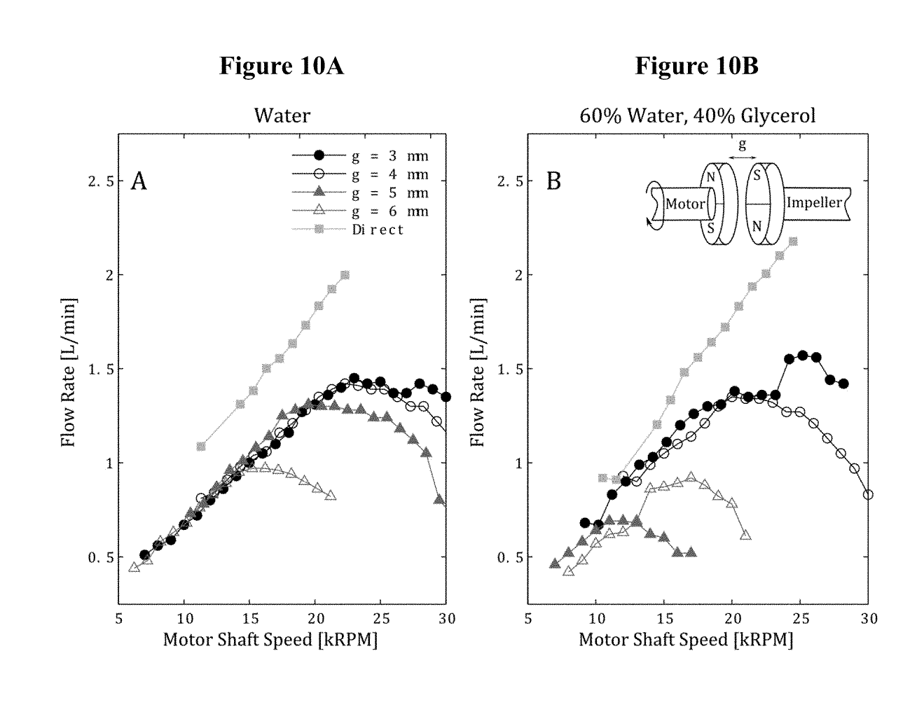

The maximum flow rate produced by the magnetically-driven CAP as a function of the motor shaft speed for different air gaps is shown in FIG. 10A. For all the curves, the flow rate increases linearly with increasing motor shaft speed up until a critical speed. Above this critical speed, flow rate drops off for increasing motor shaft speed because of slipping between the driving magnet and the impeller magnet. For instance, when the drive and impeller magnets are separated by a 3 mm gap, flow rate increases to 1.5 L/min at 22 kRPM. However, the flow rate rolls off above this motor shaft speed. Similarly, flow rate roll off occurs at 21, 16, and 15 kRPM for drive magnet and impeller magnet gaps of 4, 5, and 6 mm respectively. FIG. 10B shows the same results with blood analog as the working fluid. The pump flow rate increases linearly until 21 kRPM, for a 3 mm gap, 19 kRPM, for a 4 mm gap, and 13 kRPM for a 5 mm gap. When the impeller magnet and drive magnet are separated by 6 mm, the flow rates are shifted due to slipping between the drive and impeller magnet. For both working fluids, the flow rate produced from the magnetically driven pump is less than that produced by the direct drive CAP.

The performance of the magnetic drive CAP at different speeds in water is shown in FIG. 11A. The pressure head produced by the pump as a function of pump flow rate is displayed at various motor shaft speeds. For increasing pressure head, the flow rate produced by the pump decreases almost linearly. At 20 kRPM, the magnetic drive CAP was able to produce a maximum flow rate of 1.4 L/min and a maximum pressure head of 35 mm Hg. FIG. 11B shows the pump performance in the glycerol-water solution. At 18.5 kRPM, the magnetic drive CAP was able to produce a maximum pressure of 40 mm Hg with maximum flow rate of 1.35 L/min. In general, the pressure and flow outputs when the impeller is driven with axial flow magnets is slightly lower than with a direct drive mechanism. This is expected since there is some loss in transmission torque associated with non-contacting power transmission methods.

Axial magnetic couplings utilizing neodymium permanent magnets offer the potential to eliminate the purge seal needed in intravascular pumps like the Impella® RP, 2.5 and 5.0 pumps. This advances intravascular pump technology one step closer to fully implantable systems. FIGS. 9A and 9B demonstrate that axial magnetic couplings in the CAP design provide a one to one matching between the motor shaft speed and the impeller rotational speed up to 18 kRPM. However, FIGS. 10A and 10B reveal that while the speed is effectively transfer across the 3 mm gap separating the impeller magnet with the motor magnet, the transmitted torque is reduced. This is seen in FIG. 10B, which demonstrates that the maximum flow rate produced at 17 kRPM is 1.3 L/min with magnetic couplings separated by a 3 mm gap. When a direct drive mechanism is used, the CAP produces 1.5 L/min at 17 kRPM. This discrepancy in flow rate increases with increasing speed, which indicates the coupling is not imparting sufficient energy to the fluid. The reduction in torque transmission is confirmed by looking at the stall pressures produced by the magnetically driven CAP (FIG. 10B) and comparing to the stall pressures of the direct drive CAP (FIG. 8B). At about 17 kRPM, the magnetically driven CAP can produce 35 mm Hg pressure head while the direct drive can produce over 50 mm Hg. While there are some inefficiencies introduced when using magnetic couplings, there is room for improvement. For instance, the gap between the impeller magnet and drive magnet can still be reduced barring any limitations introduced by fabrication tolerances. Lastly, researching different magnetic coupling configurations on the intravascular pump scale can be explored. Changing the number of poles or utilizing radial magnetic couplings may provide improved torque transmission on this scale.

Even though magnetic couplings facilitate contactless torque transmission across narrow gaps, mechanical bearings are still needed to support the rotating impeller on both the inlet and outlet ends. Thus, careful consideration is needed in utilizing mechanical bearings that can support both high rotational impeller speeds and the attractive force produced between the coupling magnets. In addition, utilizing magnetic bearings necessitates small gaps between the pump housing and the rotating impeller. These narrow pathways may increase shear stress on the circulating blood, which may lead to hemolysis. Intravascular pump designs (those which are near animal testing and commercialization) that utilize magnetic couplings should be aimed at ensuring that these narrow gaps and the use of mechanical bearings do not promote blood damage. This can be studied by merging the magnetic finite element model presented in this paper with some fluid dynamics physics to estimate shear and axial forces on blood-like fluid. In addition, extensive hemolysis testing should be carried out when a pump design is nearly finalized.

While providing contactless torque transmission is necessary to eliminate the purge seal of intravascular pumps, the motor driveline still limits the use of intravascular pumps for long-term therapy. Researchers have proposed a technique to power an intravascular pump by providing a transfemoral lead that exits the patient's femoral artery (Clifton et al., The Journal of Heart and Lung Transplantation. 34(4):S177). While this technique has proven to be safe in animals, it still has the potential to lead to bleeding, infection, and thrombotic events that are traditionally associated with MCS drivelines. In addition, the study is statistically limited in the number of animals for which this method was tested. Thus, a roadmap for improving intravascular pump implantability by eliminating the purging seal system was provided. It is envisaged that the power line would exit the brachiocephalic vein with controller, backup battery and a wireless powering coil will reside in the infra-clavicular pocket, thus leveraging our prior work on wirelessly powered systems (Waters et al., ASAIO journal (American Society for Artificial Internal Organs: 1992) 2014, 60(1):31-37).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An implantable device for transferring a bodily fluid between two anatomically distinct locations in a subject, comprising:
a pump unit having an inflow port and an outflow port;
at least one anchoring structure associated with the pump unit; and
a conduit having first and second ends, the first end connected to the outflow port of the pump unit, and the second end having an outflow port.

2. The device of claim 1, wherein the pump unit has a substantially cylindrical cross section, and a diameter between about 1 mm and 20 mm.

3. The device of claim 1, wherein the pump unit comprises a motor having a motor shaft, an impeller, a casing, and a diffuser.

4. The device of claim 3, wherein the impeller is attached to the motor shaft.

5. The device of claim 3, further comprising a drive magnet and a following magnet, wherein the drive magnet is connected to the motor shaft, and the following magnet is connected to the impeller.

6. The device of claim 1, wherein the device is a catheter-deliverable cavo-arterial pump (CAP).

7. The device of claim 1, wherein the device is a catheter-deliverable right ventricular assist device (RVAD).

8. The device of claim 1, wherein the anchoring structure comprises at least a strut comprising a nonferromagnetic flexible material.

9. The device of claim 1, wherein the pump unit comprises a cable for transfer of power and data to and from the device.

10. The device of claim 1, wherein the conduit comprises an optional cannula.

11. A method of assisting right ventricular circulation in a subject, comprising:
placing the device of claim 1 in the vasculature of the subject, wherein the pump unit is anchored to the wall of the inferior vena cava (IVC) of the subject, and the outflow port of the conduit is placed in the main pulmonary artery of the subject; and
directing blood flow through the device, from the IVC of the subject to the main pulmonary artery of the subject.

12. The method of claim 11, wherein the conduit passes through the right atrium and the right ventricle of the subject.

13. The method of claim 11, wherein the pump unit comprises a motor having a motor shaft, an impeller, a casing, and a diffuser, wherein the impeller is attached to the motor shaft.

14. The method of claim 11, wherein the pump unit comprises a motor having a motor shaft, an impeller, a casing, a diffuser, a drive magnet, and a following magnet, wherein the drive magnet is connected to the motor shaft and the following magnet is connected to the impeller.

15. The method of claim 11, wherein the pump unit comprises a cable for transfer of power and data to and from the device.

16. The method of claim 11, wherein the conduit comprises an optional cannula.

17. The method of claim 11, wherein the anchoring structure comprises at least a strut comprising a nonferromagnetic flexible material.

18. The method of claim 11, wherein the blood flow is between 0 and about 5 L/min.

19. The method of claim 11, wherein the pressure head is between about 5 mmHg and about 100 mmHg.

20. The method of claim 11, wherein the impeller speed is between about 5 kRPM and 30 kRPM.
